(19) United States
(12) Patent Application Publication
(10) Pub. No.: US 2025/0067723 A1
(43) Pub. Date: Feb. 27, 2025

(54) APPARATUS AND METHODS FOR CONTROLLING INSERTION OF A MEMBRANE CHANNEL INTO A MEMBRANE

(71) Applicant: Oxford Nanopore Technologies PLC, Oxford (GB)

(72) Inventors: Benjamin James Hadwen, Oxford (GB); Michael Alexander Clifton Jennison, Oxford (GB); Max Fletcher Wilson, Oxford (GB)

(73) Assignee: Oxford Nanopore Technologies PLC, Oxford (GB)

(21) Appl. No.: 18/841,709

(22) PCT Filed: Feb. 22, 2023
(86) PCT No.: PCT/GB2023/050399

§ 371 Date: Aug. 27, 2024

(30) Foreign Application Priority Data
Feb. 28, 2022 (GB) 2202716.3

(51) Int. Cl. G01N 33/487; G01N 27/327

(52) U.S. Cl. CPC G01N 33/48721; G01N 27/327

(57) ABSTRACT

There are provided apparatuses for controlling insertion of a membrane channel into a membrane, comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid; and a driving circuit configured to apply a potential difference across the membrane via the first and second electrodes to promote insertion of a membrane channel into the membrane from the first liquid or the second liquid. Various configurations of the driving circuit are described that allow effective promotion of membrane channel insertion while reducing the risk of damage to the membrane. Corresponding methods are also described.

### RELATED APPLICATIONS

This application is a national stage filing under 35 U.S.C. § 371 of international application number PCT/GB2023/050399, filed Feb. 22, 2023, which claims the benefit of United Kingdom application number GB 2202716.3, filed Feb. 28, 2022, the entire contents of each of which are herein incorporated by reference.

### FIELD OF THE INVENTION

The invention relates to apparatus and methods for controlling the insertion of a membrane channel into a membrane. The channel may be a nanopore. The nanopore may be a protein channel.

### BACKGROUND

Channels formed across membranes can be used to sense molecular entities. Interactions between the molecular entities and the channel can cause characteristic modulations of a signal. By monitoring this signal it is possible to detect the characteristic modulations and thereby sense the molecular entities. A variety of technologies have been proposed based on this principle, such as disclosed in WO2008102120, WO2009035647, WO200079257, WO200142782 and WO2007057668. An example of such is the measurement of a current signal due to the flow of ions through a membrane channel. The membrane separates two solutions wherein the membrane channel provides a transport path through the membrane between the solutions. The membrane is highly resistive such that the sole transport pathway between the solutions is through the membrane channel or channels. The molecular entity of interest may be caused to interact with the channel, for example caused to translocate the channel.

Sensing of molecular entities using this technique provides a method of identifying single molecules and molecular entities directly, without the need for fluorescent labelling and detection. There are a wide range of possible applications, such as sequencing of DNA or other nucleic acids; sensing of chemical or biological molecules for security and defence; detection of biological markers for diagnostics; ion channel screening for drug development; and label free analysis of interactions between biological molecules.

To provide adequate throughput, an array of individual membranes may be provided wherein each membrane comprises a membrane channel.

The membrane is typically amphiphilic and may be a bilayer. Techniques for forming amphiphilic layers are well-known, such as disclosed by Montal and Mueller Proc Natl Acad Sci U S A. 1972 December; 69(12): 3561-3566, WO-2008/102120, WO2008012552 and WO2014064444. The thickness of the resultant membrane may vary due to factors such as the nature of the membrane material, solvent incorporation, the membrane geometry as well as the method of preparation.

For some systems, in the absence of an additional stimulus, membrane channels do not spontaneously insert into membranes and often insert very slowly. Selecting such a system can be advantageous as it allows control over the process of membrane insertion. Techniques to assist with insertion of membrane channels are known, the most common being the application of a potential difference across the membrane. It is thought that this potential difference stretches and thins the membrane facilitating easier insertion. Voltage assisted insertion may also be used to control the number of membrane channels that are inserted into a membrane wherein the applied potential may be actively lowered in response to membrane channel insertion and the corresponding detection of current flow through the membrane, wherein lowering of the applied potential difference following insertion of a membrane channel lowers the probability of insertion of a subsequent channel. Lowering of the applied potential may be carried out by the user which is common practise for many laboratories forming membranes and making single channel recordings. The downside to this approach is that it requires the user to observe the current of the channel (or similar observable parameter, such as resistance) and to manually react to a change in that parameter. While this is practical for single channel recordings it is impractical when dealing with a large array. It is therefore desirable to automate this process.

Various methods have been disclosed that automate the control of applied potential for an array of membranes. One uses computer control to react to the detection of an increase in the observed current flow as a consequence of nanopore insertion as disclosed by US20160289758. Another method teaches the use of an electrode mediated gas bubble to aid in membrane formation and an agitation stimulus, to achieve a similar affect, as disclosed by US20120052188. The number of channels that are required for each membrane may vary depending upon the measurement technique used. For ion flow measurements it is desirable to provide one channel per membrane.

While the use of computer controlled automation of a stimulus, such as applied potential, is beneficial over the more manual methods of controlled pore insertion, they have a number of features that are undesirable. One such feature is the need for a computer, or similar decision making circuit, to perform the operation of removing or reducing the pore insertion stimulus. Such circuits are expensive and may not be practical when dealing with a large array of membranes. Each membrane must be subject to this process of computer control to ensure good single channel yield and must either be connected to a dedicated computer control unit or must share a computer control unit between other membranes. Providing a dedicated computer control unit per channel is expensive. While connecting multiple membranes to a control unit reduces this cost that the consequence is that pore insertion is carried out in a consecutive fashion as the control unit cycles around each membrane in turn. This can increase the overall amount of time required for pore insertion across the array (selection multiplexing), or reduce the reaction time of the device (time shared multiplexing). Furthermore insertion of channels in a consecutive fashion by addition of protein nanopores to a solution in contact with the membrane has been found to result in adsorption of the nanopores to the walls of the vessel due to the increased time that the nanopores are in solution prior to being inserted. This can reduce the yield of inserted nanopores across the array.

One approach to addressing this problem is to use a voltage reduction unit. US2020179920A1 discloses “passive pore insertion” whereby a voltage applied across a membrane to perform pore insertion is adjusted by means of a voltage reduction unit placed in series with the voltage source, adjustment being in accordance with the current sourced/sunk through the membrane/pore arrangement. However, this approach is not very flexible, with many of the parameters dependent on the characteristics of the current source used.

### SUMMARY OF THE INVENTION

An approach which overcomes the problems as detailed above and which is set out in aspects of the invention is to employ a circuit element which intrinsically reduces the applied potential across the membrane when a channel inserts. In some embodiments, this does not require decision making electronics and can be deployed cheaply across a large array. Other advantages can be obtained by combining the circuit elements with other components and configurations.

According to an aspect of the invention there is provided an apparatus for controlling insertion of a membrane channel into a membrane, comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid; and a driving circuit configured to apply a potential difference across the membrane via the first and second electrodes to promote insertion of a membrane channel into the membrane from the first liquid or the second liquid, wherein: the driving circuit is configured such that: the applied potential difference prior to insertion of a membrane channel is defined by a reference voltage; and the applied potential difference after insertion of a membrane channel is defined by a selected current provided by a current source and a resistance between the first and second electrodes, a voltage limit of the current source being independent of the reference voltage, wherein the applied potential difference after insertion of the membrane channel is lower than the applied potential difference prior to insertion of the membrane channel.

The independence of the reference voltage and the voltage limit of the current source provides improved flexibility in the choice of components, while allowing the reference voltage to be chosen to fall in a range that is sufficient to promote channel insertion without damaging the membrane.

In some embodiments, the driving circuit comprises at least one power rail defining the reference voltage and at least one diode in series between the at least one power rail and the first or second electrode, the at least one power rail and at least one diode being configured to limit the applied potential difference to be no greater than the reference voltage. In some embodiments, the at least one power rail and at least one diode are configured to limit a magnitude of a voltage applied at the first electrode or the second electrode to be no greater than the reference voltage. A diode configuration is easy and cheap to implement, particularly where an array of membranes may be used. Limiting the applied potential difference ensures that the membrane should not be subjected to a voltage that would risk damaging the membrane.

In some embodiments, the driving circuit comprises a comparator configured to compare the reference voltage with a voltage at the first or second electrode, an output from the comparator determining whether the applied potential difference is defined by the reference voltage or by the selected current. A comparator is a low-cost and robust means of providing switching functionality to reduce the applied potential difference following insertion of a membrane channel.

In some embodiments, the selected current is in the range of 50-300 pA. This range of currents will result in a potential difference across the membrane that is not so large as to risk the membrane being damaged after membrane channel insertion.

In some embodiments, the driving circuit is configured to allow the selected current to be selected from a plurality of available current values, preferably by logic control. This allows the potential difference across the membrane to be selected more flexibly following membrane channel insertion.

In some embodiments, the driving circuit is configured to apply progressively increasing potential differences across the membrane. This helps to ensure only a single membrane channel is inserted into the membrane, by starting at a low potential difference that is very unlikely to cause multiple channel insertions, and increasing until a potential difference sufficient to insert a membrane channel in the specific membrane is reached.

In some embodiments, the apparatus is configured to periodically measure one or more electrical properties of the membrane and to block subsequent applying of the potential difference across the membrane by the driving circuit in response to the one or more electrical properties being indicative of a membrane channel having been inserted into the membrane. By measuring the electrical properties of the membrane, a more accurate indication can be obtained of when a membrane channel has been inserted. Blocking the potential difference after membrane channel insertion reduces the likelihood of the membrane being damaged, for example due to a high potential difference developing due to blockage of the membrane channel.

In some embodiments, the apparatus comprises a measurement circuit configured to perform the periodic measuring of the one or more electrical properties of the membrane; and the apparatus is configured to switch from a driving state to a measuring state for each measurement of the one or more electrical properties of the membrane, the driving state being such that the driving circuit is electrically connected to the first electrode or second electrode in such a way as to be able to apply the potential difference across the membrane, and the measuring state being such that the driving circuit is not electrically connected to the first electrode or second electrode in such a way as to be able to apply the potential difference across the membrane. This scheme ensures that the performance of the driving circuit is not affected by the measurement circuit, and vice versa.

In some embodiments, the apparatus is configured to block the subsequent application of the potential difference by not switching from the measuring state back to the driving state. This provides a convenient way to block the application of the potential difference to reduce the chance of damage to the membrane.

In some embodiments, the driving circuit comprises a latching arrangement configured to automatically latch the potential difference applied across the membrane, after insertion of a membrane channel into the membrane, at a value lower than the potential difference applied prior to the insertion of the membrane channel into the membrane.

According to an aspect of the invention, there is provided an apparatus for controlling insertion of a membrane channel into a membrane, comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid; and a driving circuit configured to apply a potential difference across the membrane via the first and second electrodes to promote insertion of a membrane channel into the membrane from the first liquid or the second liquid, wherein: the driving circuit is configured to automatically lower the potential difference in response to the insertion of the membrane channel into the membrane; and the driving circuit comprises a latching arrangement configured to automatically latch the potential difference applied across the membrane, after insertion of a membrane channel into the membrane, at a value lower than the potential difference applied prior to the insertion of the membrane channel into the membrane.

A latching arrangement can be used to automatically disconnect the applied potential difference after membrane channel insertion, without having to use any kind of logic control to evaluate whether to disconnect the driving circuit or the applied potential difference. This can provide the benefits of reducing the likelihood of damage to the membrane while simplifying the circuitry involved, thereby reducing cost and manufacturing complexity.

In some embodiments, the latching arrangement is configured to be triggered by a fall in the applied potential difference caused by insertion of the membrane channel into the membrane. The insertion of the membrane channel causes the resistance of the membrane (and thereby the potential difference across the membrane) to drop sharply, so this is a convenient trigger to use for the latching circuit.

In some embodiments, the driving circuit is further configured to regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

According to an aspect of the invention, there is provided an apparatus for controlling insertion of a membrane channel into a membrane, comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid; and a driving circuit configured to apply a potential difference across the membrane via the first and second electrodes to promote insertion of a membrane channel into the membrane from the first liquid or the second liquid, wherein: the driving circuit is further configured to regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

After insertion of the membrane channel, the membrane can be used for making measurements of molecules passing through the membrane channel. This process can cause depletion of a buffer solution in the first and/or second baths. Since the circuitry used to promote membrane channel insertion is configured to apply a potential difference across the membrane, it can also be used to apply a potential difference to regenerate the buffer solution. Using the same circuitry for both purposes has benefits in reducing the cost and overall complexity of the apparatus used for making measurements of molecules passing through the membrane channel.

In some embodiments, the driving circuit is configured to modulate the potential difference applied across the membrane with an AC waveform of smaller amplitude than an average amplitude of the potential difference.

According to an aspect of the invention, there is provided an apparatus for controlling insertion of a membrane channel into a membrane, comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid; and a driving circuit configured to apply a potential difference across the membrane via the first and second electrodes to promote insertion of a membrane channel into the membrane from the first liquid or the second liquid, wherein: the driving circuit is configured to modulate the potential difference applied across the membrane with an AC waveform of smaller amplitude than an average amplitude of the potential difference.

Applying a small modulation to the potential difference across the membrane can help to promote membrane channel insertion at lower average applied potential differences and/or for reduced lengths of time of applied high potential differences, thereby reducing the likelihood of damage to the membrane. Without wishing to be bound by theory, it is believed that at least a contribution to the observed effect arises due to the modulation causing disturbances or ripples leading to the small local changes in geometry and/or surface tension of the membrane that can trigger insertion.

In some embodiments, the AC waveform has an amplitude in the range of 0-100 mV and/or a frequency in the range of 0-10 kHz. These ranges of amplitudes and frequencies have been found to be particularly effective for promoting insertion of membrane channels.

In some embodiments, the apparatus is configured to: measure one or more electrical properties of the membrane; and set or adjust the potential difference applied across the membrane, before insertion of a membrane channel into the membrane, based on the measured one or more electrical properties.

According to an aspect of the invention, there is provided an apparatus for controlling insertion of a membrane channel into a membrane, comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid; and a driving circuit configured to apply a potential difference across the membrane via the first and second electrodes to promote insertion of a membrane channel into the membrane from the first liquid or the second liquid, wherein: the apparatus is configured to: measure one or more electrical properties of the membrane; and set or adjust the potential difference applied across the membrane, before insertion of a membrane channel into the membrane, based on the measured one or more electrical properties.

Setting the applied potential difference before insertion of the membrane channel based on the electrical properties of the membrane allows the potential difference to be chosen to effectively promote membrane channel insertion taking account of variations between membranes. This can improve the speed and effectiveness of membrane channel insertion by allowing the correct potential difference to be determined and applied more quickly, rather than, for instance, having to ramp up the potential difference from a low initial value.

In some embodiments, the driving circuit is configured to promote insertion of a membrane channel into a membrane simultaneously at a plurality of different pixels, each pixel being associated with first and second electrodes driven by the driving circuit. It is advantageous to provide multiple pixels to increase throughput and allow more membranes to be processed in the same amount of time.

In some embodiments, the driving circuit is configured to apply a common potential difference to pixels in which a membrane channel has not yet inserted into the membrane. This simplifies the design and operation of the apparatus with multiple pixels, because it is not necessary to individually address each pixel.

In some embodiments, the driving circuit is configured to progressively increase the common potential difference and to control the progressive increase based on detection of membrane channel insertions in other pixels. This allows an effective potential difference to be reached more rapidly by determining whether the applied potential difference is promoting membrane channel insertions over the plural pixels as a whole.

In some embodiments, the driving circuit is configured to control the progressive increase based on a detected rate of membrane channel insertion. The rate of membrane channel insertions is a useful measure of how effectively the currently applied potential difference is promoting membrane channel insertions in the plural pixels as a whole.

In some embodiments, the progressive increase comprises a series of steps and the driving circuit is configured to initiate a step to a higher potential difference when the detected rate of membrane channel insertion falls below a predetermined threshold. This progressive increase means that the potential difference is kept at a level which drives an appropriate rate of membrane channel insertion that does not increase the risk of membrane damage.

In some embodiments, the driving circuit is configured to: measure one or more electrical properties of the membrane in each of the pixels; and set or adjust the potential difference applied across the membrane in each pixel, before insertion of a membrane channel into the membrane of the pixel, based on the measured one or more electrical properties. The properties of membranes may vary, and so the appropriate potential difference to effectively promote membrane channel insertion may also differ. This allows an appropriate potential difference to be set more quickly based on the properties of the membrane, rather than having to start increasing the potential difference from a low initial value.

In some embodiments, each pixel is associated with a first electrode and a second electrode driven by the driving circuit. This provides a clear correspondence between the pixels and the first and second electrodes.

In some embodiments, two or more of the plurality of pixels are associated with respective second electrodes and share a common first electrode. By sharing a common first electrode, the structure of the apparatus and the circuitry can be simplified by allowing one common connection for applying the potential difference to the pixels.

In some embodiments, the two or more of the plurality of pixels are associated with the first bath and a plurality of respective second baths, the common first electrode being configured to contact the first liquid in the first bath, and the respective second electrodes being configured to contact the second liquid in the respective second baths. Sharing a common first bath further simplifies the structure of the apparatus, and can improve performance by allowing all the pixels to share a single first bath having greater capacity.

In some embodiments, the second electrodes are individually addressable by the driving circuit. This allows the appropriate potential difference to be provided to promote membrane channel insertion in each pixel based on the varying properties of the respective membranes while reducing the risk of damage to the membranes

In some embodiments, the pixels are arranged in a two-dimensional grid, and the second electrode associated with each pixel is connected to the second electrodes associated with other pixels on the same row or column of the grid, such that each second electrode is individually addressable using a combination of a row and column. This type of grid arrangement allows each pixel to be individually addressed with a simple structure of electrical connections.

In some embodiments, the second electrodes are each provided at the base of a respective second bath, the driving circuit is provided in an integrated circuit integral with the second bath, and the second electrodes are electrically connected to the driving circuit. Integrating the driving circuit may simplify the construction of the apparatus.

In some embodiments, the second electrodes are detachable from the driving circuit. Making the driving circuit detachable may be advantageous in devices where some components such as the second baths are disposable, so that wastage is reduced by reusing the driving circuit.

In some embodiments, the driving circuit is provided by an application-specific integrated circuit. These specialised chips can simplify manufacture and reduce manufacturing costs.

According to an aspect of the invention, there is provided a sequencing flow cell comprising an apparatus in which the driving circuit is provided by an application-specific integrated circuit, and further comprising: a control circuit configured to control the driving circuit; a sensor array configured to define the first and second baths and support the membrane; a housing comprising one or more fluid loading ports; one or more fluid channels fluidly connecting the fluid loading ports to the first and/or second baths; and an electrical connector for connecting the flow cell to a measurement instrument.

According to an aspect of the invention, there is provided a sequencing instrument comprising one or more of the apparatuses above, and further comprising: instrumentation electronics configured to interface with the one or more apparatuses; a processor configured to control the driving circuits of the one or more apparatuses via the instrumentation electronics, and to calculate a sequence of a nucleotide molecule based on measurements from the apparatuses; and optionally further comprising temperature control means configured to control the temperature of the first and second liquids in the one or more apparatuses.

In some embodiments, the one or more apparatuses are the sequencing flow cells above, the electrical connector of each of the one or more sequencing flow cells is configured to connect to the instrumentation electronics; and the processor is configured to control the driving circuits of the one or more flow cells via the control circuits of the respective flow cells.

According to an aspect of the invention, there is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises controlling a driving circuit to apply a potential difference across the membrane via the first and second electrodes to promote insertion of a membrane channel into the membrane from the first liquid or the second liquid, wherein: the applied potential difference prior to insertion of a membrane is defined by a reference voltage; and the applied potential difference after insertion of a membrane is defined by a selected current provided by a current source and a resistance between the first and second electrodes, a voltage limit of the current source being independent of the reference voltage, wherein the applied potential difference after insertion of the membrane channel is lower than the applied potential difference prior to insertion of the membrane channel.

The independence of the reference voltage and the voltage limit of the current source provides improved flexibility in the choice of components, while allowing the reference voltage to be chosen to fall in a range that is sufficient to promote channel insertion without damaging the membrane.

According to an aspect of the invention, there is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises: controlling a driving circuit to apply a potential difference across the membrane via the first and second electrodes to promote insertion of a membrane channel into the membrane from the first liquid or the second liquid, wherein: the driving circuit is configured to automatically lower the potential difference in response to the insertion of the membrane channel into the membrane; and the driving circuit comprises a latching arrangement configured to automatically latch the potential difference applied across the membrane, after insertion of a membrane channel into the membrane, at a value lower than the potential difference applied prior to the insertion of the membrane channel into the membrane.

A latching arrangement can be used to automatically disconnect the applied potential difference after membrane channel insertion, without having to use any kind of logic control to evaluate whether to disconnect the driving circuit or the applied potential difference. This can provide the benefits of reducing the likelihood of damage to the membrane while simplifying the circuitry involved, thereby reducing cost and manufacturing complexity.

According to an aspect of the invention, there is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises controlling a driving circuit to: apply a potential difference across the membrane via the first and second electrodes to promote insertion of a membrane channel into the membrane from the first liquid or the second liquid; and regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

After insertion of the membrane channel, the membrane can be used for making measurements of molecules passing through the membrane channel. This process can cause depletion of a buffer solution in the first and/or second baths. Since the circuitry used to promote membrane channel insertion is configured to apply a potential difference across the membrane, it can also be used to apply a potential difference to regenerate the buffer solution. Using the same circuitry for both purposes has benefits in reducing the cost and overall complexity of the apparatus used for making measurements of molecules passing through the membrane channel.

According to an aspect of the invention, there is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises controlling a driving circuit to apply a potential difference across the membrane via the first and second electrodes to promote insertion of a membrane channel into the membrane from the first liquid or the second liquid; and modulating the potential difference applied across the membrane with an AC waveform of smaller amplitude than an average amplitude of the potential difference.

Applying a small modulation to the potential difference across the membrane can help to promote membrane channel insertion at lower average applied potential differences and/or for reduced lengths of time of applied high potential differences, thereby reducing the likelihood of damage to the membrane. Without wishing to be bound by theory, it is believed that at least a contribution to the observed effect arises due to the modulation causing disturbances or ripples leading to the small local changes in geometry and or surface tension of the membrane that can trigger insertion.

According to an aspect of the invention, there is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises: controlling a driving circuit to apply a potential difference across the membrane via the first and second electrodes to promote insertion of a membrane channel into the membrane from the first liquid or the second liquid measuring one or more electrical properties of the membrane; and setting or adjusting the potential difference applied across the membrane, before insertion of a membrane channel into the membrane, based on the measured one or more electrical properties.

Setting the applied potential difference before insertion of the membrane channel based on the electrical properties of the membrane allows the potential difference to be chosen to effectively promote membrane channel insertion taking account of variations between membranes. This can improve the speed and effectiveness of membrane channel insertion by allowing the correct potential difference to be determined and applied more quickly, rather than, for instance, having to ramp up the potential difference from a low initial value.

### BRIEF DESCRIPTION OF THE DRAWINGS

Embodiments of the invention will now be described, by way of a non-limiting example only, with reference to the accompanying drawings in which corresponding reference symbols indicate corresponding parts, and in which according to some embodiments of the disclosure:

FIG. 1 shows an apparatus according to an embodiment;

FIG. 2 shows an apparatus according to an embodiment, showing an exemplary layout of pixels in a grid arrangement;

FIG. 3 is an electrical diagram of the apparatus with an electrical representation of the membrane;

FIG. 4 is a circuit diagram of one implementation of the pore insertion circuit;

FIG. 5 shows the behaviour of the voltage applied to the second electrode by the circuit of FIG. 4 as a function of the load resistance;

FIG. 6 is a circuit diagram of an alternative implementation of the pore insertion circuit using a comparator;

FIG. 7 is a circuit diagram of an alternative implementation of the pore insertion circuit using a comparator and able to output plural values of the selected current;

FIGS. 8a and 8b show an implementation of increasing the reference voltage and selected current by ramping;

FIGS. 9a and 9b show an implementation of increasing the reference voltage and selected current by stepping;

FIG. 10 shows stepping of the reference voltage in response to the apparatus detecting a fall in pore insertion rate;

FIG. 11 shows switching between the pore insertion circuit and measurement circuit according to an embodiment;

FIG. 12 shows stepping of the reference voltage as an alternative to the ramping of the reference voltage shown in FIG. 11;

FIG. 13 shows a flow diagram of an example embodiment where the potential difference applied to the membrane prior to membrane channel insertion is set according to measurements of the electrical properties of the membrane;

FIG. 14 is a circuit diagram of a pore insertion circuit in which a latching arrangement latches the value of the potential difference following insertion of a membrane channel;

FIG. 15 is a schematic of an embodiment in which the apparatus is also configured to regenerate a mediator;

FIG. 16 shows the current supplied by the pore insertion circuit before and after membrane channel insertion, during a sequencing measurement, and during mediator regeneration;

FIG. 17 is a circuit diagram of an implementation in which the pore insertion circuit is further configured to regenerate a mediator;

FIG. 18 is a schematic of an embodiment in which the driving circuit applies a potential difference modulated by an AC waveform;

FIG. 19 shows an example of an AC waveform applied to the first electrode in the embodiment of FIG. 18;

FIG. 20 is a schematic of an apparatus according to an embodiment being used as part of a sequencing flowcell;

FIG. 21 is a schematic of a sequencing instrument using a sequencing flowcell comprising an apparatus according to an embodiment;

FIG. 22 is a picture of an exemplary sequencing flowcell product in which the apparatus may be implemented;

FIG. 23 is a picture of an exemplary sequencing instrument in which the sequencing flowcell comprising the apparatus may be used;

FIG. 24 is a picture of an exemplary sequencing instrument in which the sequencing flowcell comprising the apparatus may be used; and

FIG. 25 is a picture of an exemplary sequencing instrument in which the sequencing flowcell comprising the apparatus may be used.

### DETAILED DESCRIPTION OF THE INVENTION

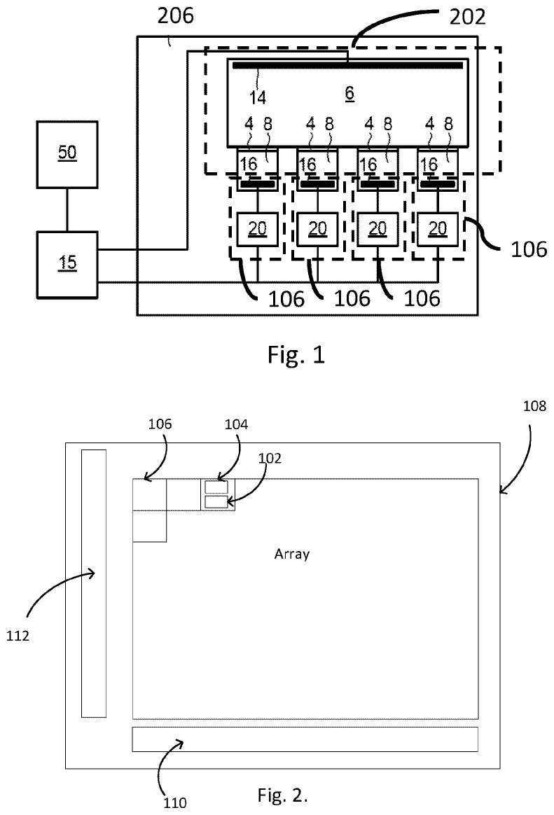

FIG. 1 depicts an apparatus for controlling insertion of a membrane channel into a membrane 4 according to an embodiment. The apparatus of FIG. 1 may also be used for single molecule detection by nanopore sensing. As described above, the membrane 4 is typically amphiphilic and may be a bilayer. The membrane channel to be inserted into the membrane 4 may be any suitable membrane channel and may also be referred to as a pore or a nanopore.

The apparatus comprises a housing 206, and comprises a first bath 6 contained in the housing 206. The first bath 6 holds a first liquid. The first liquid contacts a first surface of each membrane 4.

The apparatus further comprises four second baths 8 (also referred to as wells) contained in the housing 206. A different number of second baths 8 may be provided in other embodiments. Each second bath 8 is sealed by a membrane 4.

Each second bath 8 holds a second liquid. The second liquid in each second bath 8 contacts a second surface of the membrane 4 sealing that second bath 8. Thereby, the membrane separates the first and second liquids. Typically the membrane thickness is within the range of 1 nm to 5 nm.

A first electrode 14 provided in the first bath 6 is configured to contact the first liquid. A second electrode 16 is configured to contact the second liquid in each second bath 8. Each second bath 8 has its own second electrode 16. Being configured to contact the liquids means that the first and second electrodes 14, 16 make at least electrical contact with the first and second liquids respectively. This may be achieved by the first and second electrodes 14, 16 being in direct contact with the first and second liquids. Alternatively, the first and second electrodes 14, 16 may make electrical contact with the first and second liquids via an intermediate layer such as an oil or lipid layer.

The first and second electrodes 14, 16 can be used to control the insertion of a membrane channel into the membrane 4, as described below. In certain embodiments, at a later time after the membrane channel has been inserted, the first and second electrodes 14, 16 can also be used to sense molecular entities via their interaction with the membrane channel.

The apparatus further comprises a driving circuit configured to apply a potential difference across the membrane 4 (which may also be referred to as a membrane voltage) via the first and second electrodes 14, 16. The applied membrane voltage can be such as to promote insertion of a membrane channel into the membrane 4 from the first liquid 6 or the second liquid 8. Membrane channels (which may comprise membrane proteins for example) are thus provided in the first liquid or the second liquid or both. The membrane voltage promotes insertion of the membrane channel. As mentioned in the introductory part of the description and without wishing to be bound by theory, it is thought that the voltage assists insertion by stretching and thinning the membrane 4.

The housing 206 is shown with a single first bath 6 and four second baths 8 with associated membranes 4, although this is not essential and only a single first bath 6 and second bath 8 may be provided. In other embodiments, many more first baths 6 and second baths 8, and corresponding first electrodes 14 and second electrodes 16, may be provided. The number of second baths may be any integer between 1 and 100,000. It may be 100 or more, 1000 or more or 10,000 or more.

Each second bath 8 and second electrode 16 may be considered to be part of a pixel 106. The driving circuit is configured to promote insertion of a membrane channel into the membrane 4 simultaneously at a plurality of different pixels 106. Each pixel 106 is associated with a first electrode 14 and a second electrode 16 driven by the driving circuit. The driving circuit comprises pixel circuitry 20, which is specific to and replicated between each pixel 106, and some common circuitry 15 that is shared between some or all of the pixels 106. The apparatus may also comprise control circuitry 50 used to control the driving circuit.

In some embodiments, each pixel 106 is associated with a respective pair of first and second electrodes 14, 16 driven by the driving circuit. However, more preferably, and as shown in FIG. 1, two or more of the plurality of pixels 106 are associated with respective second electrodes 16 and share a common first electrode 14. In FIG. 1, four pixels 106 are associated with the common first electrode 14. The two or more of the plurality of pixels 106 are associated with the first bath 6 and a plurality of respective second baths 8. The common first electrode 14 is configured to contact the first liquid in the first bath 6, and the respective second electrodes 16 are configured to contact the second liquid in the respective second baths 8. This allows multiple pixels to share a single first bath 6 and first electrode 14.

In some embodiments, such as that in FIG. 1, the second electrodes 16 are each provided at the base of a respective second bath 8. The driving circuit may be provided in an integrated circuit integral with the second bath 8. The second electrodes 16 may be electrically connected to the driving circuit, i.e. the second electrodes 16 may form a part of the integrated circuit in which the driving circuit is provided. Alternatively, the second electrodes 16 may be detachable from the driving circuit. This may be advantageous in some situation, for example if the second bath 8 and second electrode 16 are provided as part of a disposable component, and the driving circuit is to be reused between multiple disposable components.

Preferably, the second electrodes 16 are individually addressable by the driving circuit, for example via the corresponding pixel circuitry 20. This means that the insertion of a membrane channel into each membrane 4 can be controlled for each pixel 106, thereby reducing the chance of inserting multiple membrane channels into the same membrane 4, or of causing damage to any membrane 4.

The driving circuit may be provided by an application-specific integrated circuit (ASIC) 108. Other parts of the electronics and electrical connections within the apparatus may also be provided by the ASIC 108, such as the second electrodes 16.

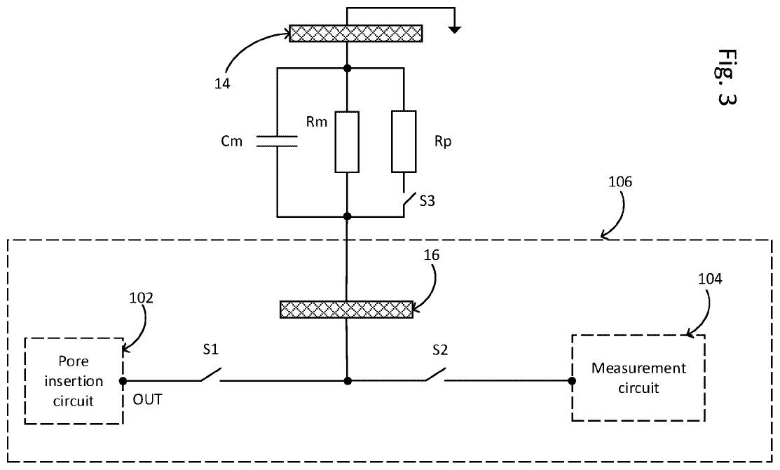

FIG. 2 shows a high-level layout of an exemplary design of such an ASIC 108. FIG. 2 shows an embodiment in which the pixels 106 are arranged in an array comprising a two-dimensional grid, which allows them to be individually addressed.

As mentioned above, the size of the array on the ASIC 108 (i.e. the number of pixels 106) will be application dependent. For example, the size may depend on the quantity of sequencing data to be measured by a sequencing instrument in which the apparatus is to be used. The array may comprise for example 100 pixels, 1000 pixels, 10,000 pixels or 100,000 pixels or more. In the examples shown herein, each pixel 106 is associated with a single second bath 8 and second electrode 16. However, in some embodiments, multiple second baths 8 and second electrodes 16 may be connected to each pixel, for example via an isolation switch, as described in WO2010/122293A1.

The second electrode 16 associated with each pixel 106 is connected to the second electrodes 16 associated with other pixels 106 on the same row or column of the grid. This connection may be achieved using circuitry 110, 112 for addressing and data readout of the pixels 106 on each row and column. Thereby, each pixel 106 and the second electrode 16 therein is individually addressable using a combination of a row and column. Similarly as shown in FIG. 1, the first electrode 14 may be shared between all pixels 106 in the array, or at least some groups of pixels 106 in the array may share a first electrode 14.

The size of each pixel 106 may be in the range 40 μm-400 μm, or more typically in the range of 200 μm to 300 μm. This will depend on factors such as the desired form factor of the overall product, the number of pixels, the configuration of a sensor array 202, and the design of the circuitry of the ASIC 108.

The ASIC 108 may further comprise additional circuitry for programming voltages and currents set within each pixel 106, for example the reference voltage and selected current. Circuitry may also be included for digitisation and readout of signals measured from each pixel 106, and multiplexing of the outputs from multiple pixels 106 into a format convenient for readout off the ASIC. Further description of exemplary ASIC configurations may be found in WO2016/181118A1.

The ASIC 108 may further comprise a means for measuring the temperature of all or part of the apparatus, for example the temperature of the first and second liquids in the first and second bath 6, 8. The ASIC 108 may further comprise a means for heating and/or cooling all or part of the apparatus, for example for heating and/or cooling the temperature of the first and second liquids in the first and second bath 6, 8. The ASIC may further comprise non-volatile memory and the means to read from/write to it. Such memory may be used, for example to store and recall calibration or test information, information regarding electrical properties associated with each pixel 106 (for example the electrical properties of the membrane 4) and associated sensor array structures above it, information regarding Lot IDs, information relating to use, such as measurement data, measurement conditions (time and date, measured temperature, user input data, etc.), error codes.

Each pixel 106 comprises a measurement circuit 104 for measuring an electrical signal (typically current) comprising sequencing data, and a pore insertion circuit 102 for supplying electrical signals to promote the insertion of a pore into the membrane. The pixel 106 may further comprise (not shown) circuitry for a number of additional functions including calibration, self-test, reverse bias modes for unblocking and mediator regeneration.

FIG. 3 shows an electrical representation of the arrangement for pore insertion at each pixel 106 of the nanopore array. Each pixel 106 has an associated second electrode 16, to which is connected the pixel circuitry 20, which comprises a measurement circuit 104, and a pore insertion circuit 102.

The measurement circuit 104 and pore insertion circuit 102 may configured to be disconnected from the second electrode 16 by means of switches S2 and S1 respectively. The electrical properties of the membrane 4 and its environ are represented by capacitor Cₘ and resistor Rₘ respectively. Rₚ represents the resistance of the nanopore (membrane channel), the insertion of which into the membrane 4 may be represented electrically by closing switch S3. The first electrode 14 may be common to some or all pixels 106 in the array, as discussed above.

The potential difference across the membrane may be controlled by varying the voltage at one or both of the first and second electrodes 14, 16. However, preferably a voltage at the first electrode 14 is held constant, and the applied potential difference is controlled by controlling a voltage at the second electrode 16. For example, the first electrode 14 may be connected to a DC bias supply. The bias supply holds the voltage at the first electrode 14 at a fixed level, e.g. 0V.

Various circuit configurations of the pore insertion circuit 102 will now be described in accordance with embodiments.

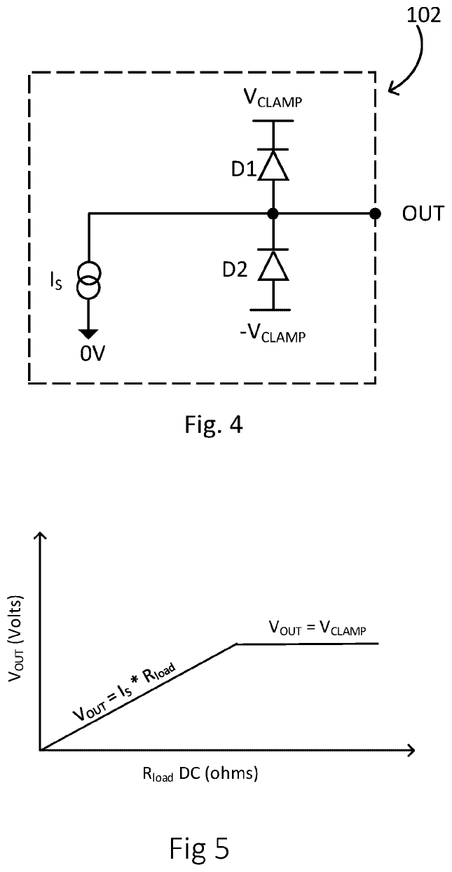

A first example of the pore insertion circuit 102 is shown in FIG. 4. The pore insertion circuit 102 comprises a current source. This may be any suitable current source of standard design, and is configured to source a current I_{S}. Using this design of the pore insertion circuit 102, the driving circuit is configured such that the applied potential difference prior to insertion of a membrane channel is defined by a reference voltage. The applied potential difference after insertion of a membrane channel is defined by a selected current provided by a current source and a resistance between the first and second electrodes 14, 16. The driving circuit is configured such that a voltage limit of the current source is independent of the reference voltage, and the applied potential difference after insertion of the membrane channel is lower than the applied potential difference prior to insertion of the membrane channel.

This can be achieved by the driving circuit comprising at least one power rail defining the reference voltage and having at least one diode D1, D2 in series between the at least one power rail and the first or second electrode 14, 16. The at least one power rail and at least one diode D1, D2 are configured to limit the applied potential difference to be no greater than the reference voltage. The power rail can be implemented using, for example, a DC power supply or a connection thereto. The diode D1, D2 can be any component that will act as a diode when connected in an appropriate manner. For example, D1 and D2 in FIG. 4 could be implemented using a normal two-terminal diode, but could also be implemented using a transistor. In the case of the transistor, the gate/base and emitter of the transistor could be used as the two diode terminals.

The at least one power rail and at least one diode D1, D2 may be configured to limit a magnitude of a voltage applied at the first electrode 14 or the second electrode 16 to be no greater than the reference voltage.

In the embodiment of FIG. 4, a pair of diodes D1, D2 are arranged in parallel at the output of the current source. The diodes are arranged to clamp the output voltage generated by the pore insertion circuit 102 (V_{OUT}) to a level that is between VCLAMP and −VCLAMP, where V_{CLAMP} represents the reference voltage. When not clamped at either extreme, the output voltage is determined by the selected current from the current source and the DC load resistance, i.e. the resistance between the first and second electrodes 14, 16.

Overall, the circuit acts as either a voltage source or a current source, in accordance with the DC load impedance presented at its output. In the embodiment of FIG. 3, the voltage V_{OUT} is applied to the second electrode 16. However, as discussed above it could be applied to the first electrode 14 in alternative embodiments.

In FIG. 4, the output voltage V_{OUT} is given by:

V_{OUT}=I_{S}*R_{load} (for I_{S}*R_{load}<V_{CLAMP})

V_{OUT}=V_{CLAMP} (for I_{S}*R_{load}>V_{CLAMP})

The output current I_{OUT} is given by:

I_{OUT}=I_{S} (for I_{S}*R_{load}<V_{CLAMP})

I_{OUT}=V_{CLAMP}/R_{load} (for I_{S}*R_{load}>V_{CLAMP})

The behaviour of V_{OUT} as a function of the DC load is shown in FIG. 5. This illustrates the two operating modes of the circuit, whereby the output is either a constant at the reference voltage (V_{CLAMP}) or a constant selected current (I_{S}).

The value of the reference voltage V_{CLAMP} may be chosen in accordance with the properties and/or materials of the membrane channel and/or membrane to provide favourable conditions for pore insertion. For example, the value of the reference voltage V_{CLAMP} may be between 0.2V and 0.6V, optionally between 0.3V and 0.5V, optionally around 0.4V.

The value of the selected current I_{S} may be chosen such that I_{S}*Rₘ>V_{CLAMP}, ensuring that the circuit is in voltage limited mode (output V_{CLAMP}) prior to a membrane channel being inserted. However, the selected current should also be chosen to be not so large that I_{S}*RₘRₚ/(Rₘ+Rₚ) is a sufficiently high voltage so as to place the membrane under significant stress, whereby there is a risk of it bursting.

The optimum value of I_{S} thus depends on other system parameters (V_{CLAMP}, Rₘ, Rₚ). For a typical system I_{S} might be in the range 50-300 pA, optionally 50-250 pA, optionally 100-150 pA, optionally around 130 pA. Membrane channel resistances may typically be in the range of 0.1-10 GOhms, more typically 0.5-2 GOhms, even more typically above or around 1 GOhm.

In certain embodiments, the design of the integrated circuitry may facilitate V_{CLAMP} and I_{S} to be set differently for different pixels in the array. In other embodiments V_{CLAMP} and I_{S} may be globally set for the whole array. For example, some or all pixels 106 may share their pixel circuitry 20 and the corresponding pore insertion circuit 102.

An advantage of this embodiment is its simplicity to implement, requiring few circuit components, and low power consumption. This makes it well suited to miniaturisation in pixels of a small size.

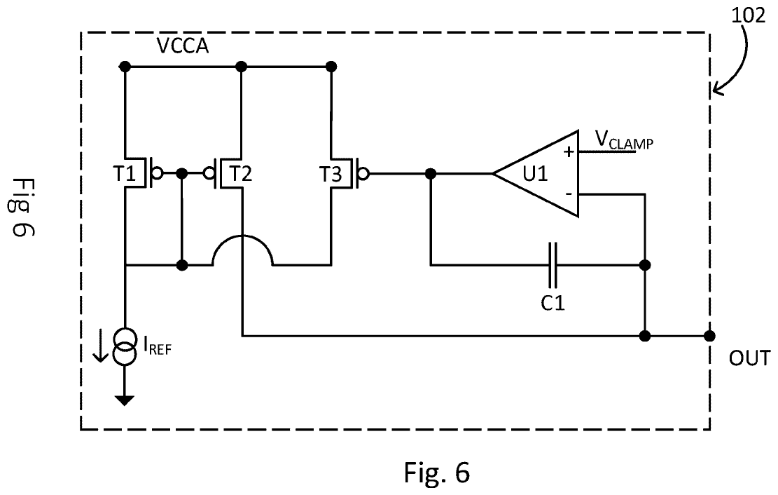

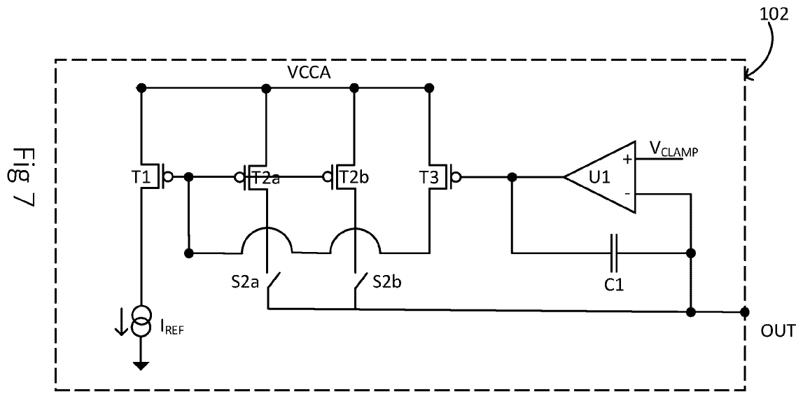

An alternative circuit arrangement for the pore insertion circuit 102 is shown in FIG. 6. In this embodiment, the driving circuit comprises a comparator U1 configured to compare the reference voltage with a voltage at the first or second electrode 14, 16. An output from the comparator U1 determines whether the applied potential difference is defined by the reference voltage or by the selected current. The reference voltage may be applied to one of the inputs of the comparator. Each transistor has one terminal connected to the supply rail VCCA.

In FIG. 6, U1 is a comparator whose output is high when the voltage at the positive input (+) exceeds that of the negative input (−), and low when the voltage at (+) is less than that at (−). The comparator U1 may be any conventional comparator component. In some embodiments, the comparator U1 may comprise an op-amp and the reference voltage is applied to one of the inputs of the op-amp. Similar to the embodiment of FIG. 4, the circuit in FIG. 6 is configured to function as a current source, whose output voltage is clamped to V_{CLAMP}.

In current source mode (after insertion of a membrane channel), V_{OUT}<V_{CLAMP}. This means that the output of the comparator U1 is high. Therefore T3 is turned off so the gates of T1 and T2 are low and these transistors are turned on. The transistor pair T1 and T2 are configured to function as a current mirror, of standard design, so that the current through T2 is the reference current I_{REF} multiplied by the relative sizes (W/L ratios) of T1 and T2=I_{S}, and this current is sourced through the output.

In voltage mode (before insertion of a membrane channel), V_{OUT} tends to V_{CLAMP}, and the output of the comparator U1 is low. Therefore, T3 is turned on so the gates of T1 and T2 are high and these transistors are turned off. The value of V_{OUT} is maintained at V_{CLAMP} by the feedback loop 5 of the comparator U1.

FIG. 7 shows a variant of the embodiment of FIG. 6, in which the driving circuit is configured to allow the selected current to be selected from a plurality of available current values. In FIG. 7, the output current in current mode (after insertion of a membrane channel) is selectable according to one of three values.

The selected current is preferably selected by logic control. In FIG. 7, the logic control (not shown) is arranged so that either or both of switches S2a, S2b are closed. The current mirror is thus configured so that the output device is either T2a, T2b or both T2a and T2b in parallel. Thus the output current sourced though OUT is selectable between three values according to the configuration of switches S2a and S2b.

It will be appreciable to one of ordinary skill in the art how the choice of different selected currents in current mode (after insertion of a membrane channel) may be determined by adding additional load devices T2 in parallel and enabling different configurations of T2 to be connected between the rail VCCA and OUT by control of appropriate switches.

An advantage of the embodiment of FIG. 7 (e.g. compared to the embodiment of FIG. 6) is that V_{CLAMP} can be defined accurately and is not subject to transistor process variations. Variations in the characteristics of the transistors due to process variations may mean that the exact voltage across different transistors may vary slightly for the same selected current. Therefore to ensure the voltage across the membrane is always at V_{CLAMP} when desired, it may be necessary to use a higher selected current than is ideal to account for possible variations in the transistor properties. The embodiment of FIG. 7 allows for selection between different transistors, or effectively to average the transistor properties, with different selections of S2a and S2b. This allows the selected current to be set to more accurately and reliably apply the reference voltage V_{CLAMP}.

The driving circuit may be configured to apply a common potential difference to pixels 106 in which a membrane channel has not yet inserted into the membrane. The voltage at which membrane channel insertion happens varies from membrane 4 to membrane 4. Different membranes 4 are found to behave differently according to natural production variations of membrane thickness and capacitance. Therefore, the probability that a pore inserts into the membrane 4 (within a given period of time) has a different voltage dependence for different membranes. This may be exploited in driving circuit embodiments by allowing for the value of V_{CLAMP} to vary over time. The driving circuit 15 may therefore be configured to increase the membrane voltage (common potential difference) progressively until insertion occurs.

In any of the embodiments of FIGS. 4, 6, and 7, the driving circuit may be configured to apply progressively increasing potential differences across the membrane. It is advantageous to adjust the value of the output V_{OUT} of the pore insertion circuit 102 over time for performing pore insertion. This may comprise progressively increasing the reference voltage defining the applied potential difference prior to insertion of a membrane channel.

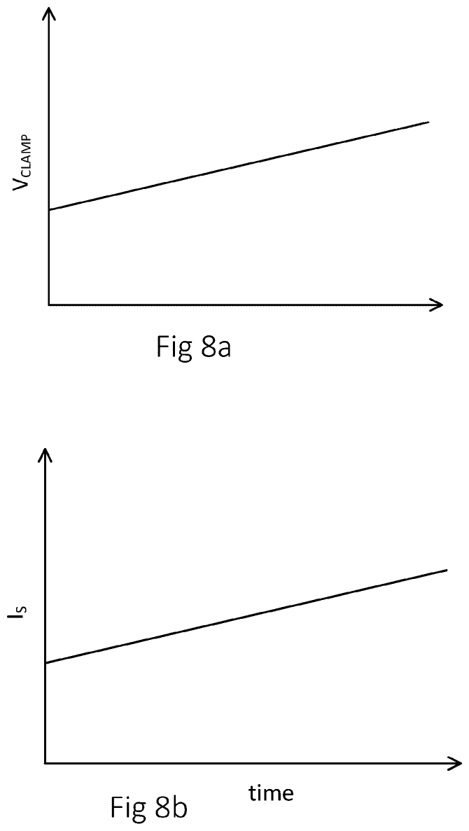

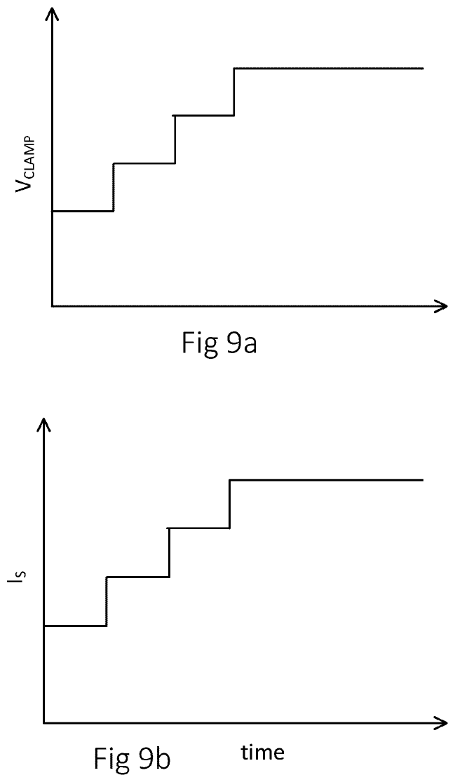

FIGS. 8a and 9a show two possible embodiments, whereby the reference voltage V_{CLAMP} is gradually increased using a ramp (FIG. 8a) or step (FIG. 9a) function.

An advantage of this embodiment is that a high proportion of membranes 4 may successfully have a single pore inserted into them. Starting at low V_{CLAMP}, membranes 4 that are thinner are more likely to have a pore inserted at lower V_{CLAMP} and without popping. Changing the applied potential difference to a lower value (e.g. by disconnecting the reference voltage V_{CLAMP}) following pore insertion reduces the risk of the membrane 4 subsequently popping (due to the membrane potential that is created as a consequence of I_{S} being sourced through the membrane). It also reduces the chance of an unwanted second pore inserting into the membrane 4.

Changing the applied potential difference to a lower value after membrane channel insertion also reduces the risk of a high voltage being applied across the membrane 4 suddenly and unexpectedly in the event that the membrane channel subsequently becomes fully or partially blocked. Such a blockage would abruptly increase the membrane channel resistance and cause the voltage developed across the membrane 4 to increase. This may be particularly problematic if the reference voltage has been increased in the period between pore insertion and pore blockage to insert pores in other membranes 4, such that the abruptly increased voltage is above what is appropriate for the particular membrane 4 that has become blocked. A further advantage of this embodiment is that it reduces the set-up time required to perform pore insertion on a target proportion of membranes 4 in an array.

In a further refinement of this embodiment, the current I_{S} may also be ramped or stepped over time, as shown in FIG. 8b and FIG. 9b.

The timings associated with steps in the reference voltage V_{CLAMP} could be determined as a function of the measured number of pore insertions. In other words, the driving circuit may be configured to control the progressive increase of the applied potential difference based on detection of membrane channel insertions in other pixels 106. This means that the potential difference applied to each pixel 106 before membrane channel insertion may be based at least in part on the effectiveness of the present value of the potential difference in promoting channel insertions.

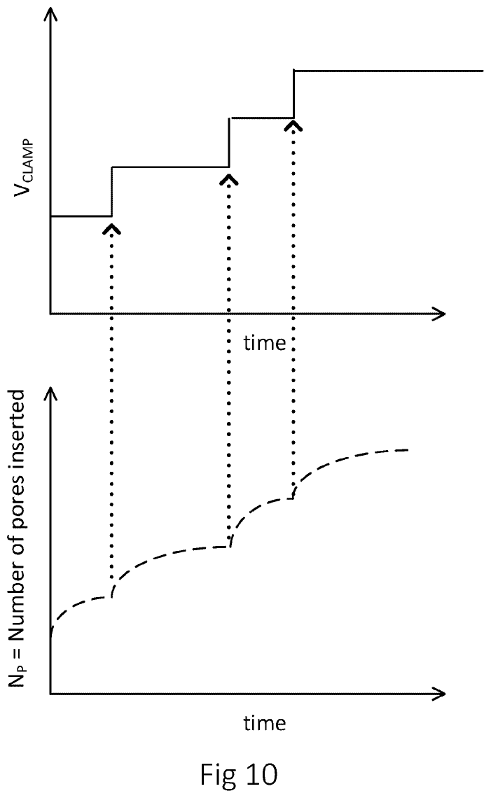

This is shown in FIG. 10. A value of V_{CLAMP} is set, and the number of pores successfully inserted is monitored at discrete steps in time. In particular in FIG. 10, the driving circuit is configured to control the progressive increase based on a detected rate of membrane channel insertion. Once the number N_{P} of channel insertions appears to saturate (i.e. dN_{P}/dt becomes small), the value of V_{CLAMP} is stepped to the next value). Where the progressive increase comprises a series of steps, the driving circuit may be configured to initiate a step to a higher potential difference when the detected rate of membrane channel insertion falls below a predetermined threshold. Alternatively, other schemes may be employed. For example, the driving circuit may be configured to ramp the applied potential difference more quickly when the detected rate of membrane channel insertion is below a predetermined threshold.

This embodiment has the advantage of improving pore insertion inefficiency whilst simultaneously reducing the amount of time needed to insert pores into a high proportion of membranes 4 in the array. By controlling the applied potential difference to pixels 106 in which a membrane channel has not yet inserted based on membrane channel insertions in other pixels 106, in particular based on the rate of channel insertions, a more consistent rate of channel insertions can be achieved. This will allow for successful channel insertions in a desired proportion of the membranes 4 in the array in a shorter time with a lower risk of damage to any particular membrane 4.

In another embodiment in which the driving circuit is configured to apply progressively increasing potential differences across the membrane 4, the membrane 4 may periodically be monitored to see if a pore has been inserted, for example by measuring its DC impedance. In such an embodiment, the apparatus is configured to periodically measure one or more electrical properties of the membrane 4 and to block subsequent applying of the potential difference across the membrane 4 by the driving circuit in response to the one or more electrical properties being indicative of a membrane channel having been inserted into the membrane 4.

For example, in the example of FIG. 3, the apparatus comprises a measurement circuit 104 configured to perform the periodic measuring of the one or more electrical properties of the membrane 4. The apparatus is configured to switch from a driving state to a measuring state for each measurement of the one or more electrical properties of the membrane 4. This can be achieved using the switches S1 and S2.

The driving state is such that the driving circuit is electrically connected to the first electrode 14 or second electrode 16 in such a way as to be able to apply the potential difference across the membrane 4. The measuring state is such that the driving circuit is not electrically connected to the first electrode 14 or second electrode 16 in such a way as to be able to apply the potential difference across the membrane 4. For example in FIG. 3, the driving state is a state in which switch S1 is closed, and the measuring state is a state in which switch S1 is open.

Preferably, in the driving state, the measurement circuit 104 is not electrically connected to the first or second electrodes 14, 16, such that the measurement circuit 104 does not affect the application of the potential difference to the membrane by the driving circuit. In FIG. 3, this can be achieved by opening switch S2 in the driving state. Switch S2 would then be closed in the measurement state such that the measurement circuit 104 can perform the periodic measuring of the one or more electrical properties of the membrane 4.

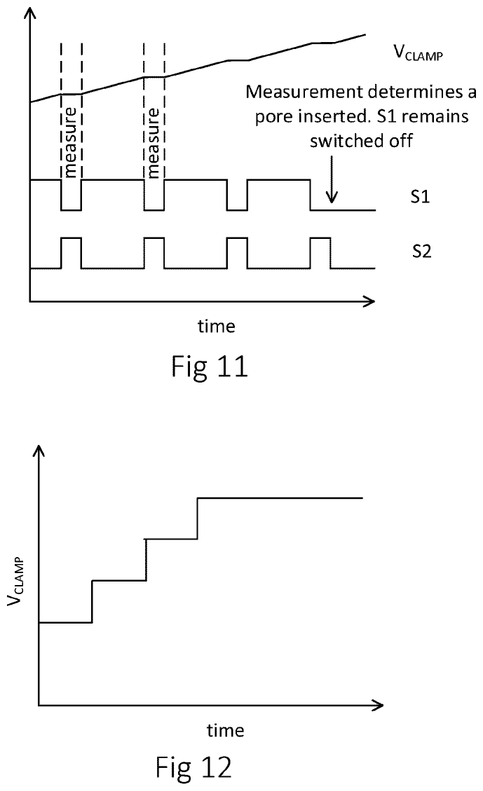

FIGS. 11 and 12 demonstrate an implementation of this embodiment. Periodically, for example every 0.1 s, 0.5 s, 1 s, or 5 s, the apparatus switches from the driving state to the measurement state. In the measurement state, pore insertion circuit 102 is disconnected from the membrane 4 by opening switch S1 and the measurement circuit 104 is connected by closing switch S2. The DC current through the membrane is measured by the measurement circuit 104, thus determining whether a pore has successfully inserted into the membrane 4.

Preferably, the apparatus is configured to block the subsequent application of the potential difference by not switching from the measuring state back to the driving state. In the example of FIGS. 3, 11, and 12, when successful pore insertion is detected, switch S1 is configured to remain open for the remainder of the pore insertion procedure.

In combination with the progressive increase of the applied potential voltage, this embodiment provides that pore insertion is attempted first at smaller values of the reference voltage V_{CLAMP}. If pore insertion is successful, the pore insertion circuit 102 becomes disconnected, thereby preventing damage to the membrane from the continued application of the potential difference. If pore insertion is not successful, it is re-attempted periodically with gradually increasing values of the reference voltage V_{CLAMP} (either ramped as in FIG. 11 or stepped as in FIG. 12).

Additionally or alternatively to the embodiments described above, measurements of the one or more electrical properties of the membrane 4 can be used to determine an appropriate reference voltage. In an embodiment, the driving circuit is configured to measure one or more electrical properties of the membrane. The driving circuit may measure one or more electrical properties of the membrane 4 in each of the pixels 106 where there are multiple pixels. This could be achieved using the measurement circuit 104.

The driving circuit is then configured to set or adjust the potential difference applied across the membrane, before insertion of a membrane channel into the membrane, based on the measured one or more electrical properties. Where there are multiple pixels, the driving circuit may set or adjust the potential difference applied across the membrane 4 in each pixel 106, before insertion of a membrane channel into the membrane 4 of the pixel 106, based on the measured one or more electrical properties. An advantage this approach is that the electrical conditions created by the driving circuit to promote pore insertion may be adapted according to membrane characteristics. These could include different material characteristics of the membrane or the pore, or in accordance with chip to chip or pixel to pixel variability associated with manufacturing. This may allow the reference voltage applied across the membrane for each pixel 106 to be more rapidly set to a value that is likely to effectively promote pore insertion.

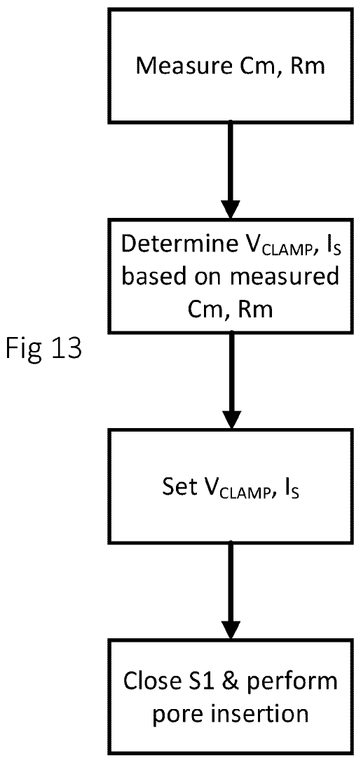

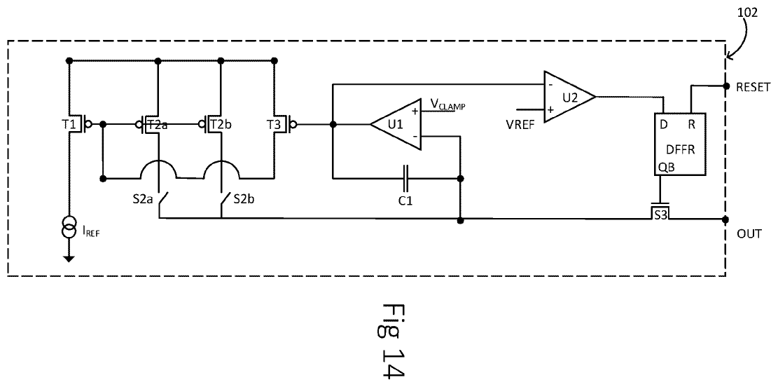

This approach may be implemented in, for example, the embodiments shown in FIG. 4, 6 or 7. The values of the reference voltage V_{CLAMP} and/or the selected current I_{S} used for performing pore insertion may be selected in accordance with the measured properties of the membrane 4, e.g. its capacitance Cₘ and resistance Rₘ. The workflow is shown schematically in FIG. 13.

With reference to FIG. 3, the electrical properties of the membrane 4 may be determined by switching the apparatus into the measurement state by closing switch S2 and opening switch S1. The electrical properties of the membrane, such as Cₘ and Rₘ can then be measured with the measurement circuit 104.

Appropriate electrical parameters, such as the values of the reference voltage and selected current associated with the pore insertion circuit 102 may then be defined according to the measured values of Cm and Rm, globally or on a pixel-by-pixel basis.

In one embodiment as shown in FIG. 7, the reference voltage V_{CLAMP} and/or the selected current I_{S} are selectively programmed on a pixel-by-pixel basis. Alternatively, the reference voltage V_{CLAMP} and/or the selected current I_{S} are globally defined common to all pixels 106. The reference voltage V_{CLAMP} and/or the selected current I_{S} may be stepped between different values, with different configurations of switch S2 opened at different steps.

Variations in the membrane resistance Rₘ, which represents leakage paths through or around the membrane 4, may influence the choice of current I_{S}, since the value of Rₘ may determine whether the pore insertion circuit 102 is operating so as to limit the voltage or limit the current (i.e. in the voltage limited mode or the current limited mode). Typically, the value of I_{S} may be chosen such that I_{S}*Rₘ exceeds the clamp (reference) voltage V_{CLAMP} (so that the driving circuit applies V_{CLAMP} across the membrane before pore insertion occurs), but not so large that a significant potential is developed across the membrane 4 after the pore is inserted. This reduces the risk of damage to the membrane 4 after membrane channel insertion. Depending on the configuration and size of the pixel 106, typical values of I_{S} may be in the range 0-300 pA, optionally in the range 100-200 pA, optionally around 150 pA.

Alternatively or additionally to the embodiments described above, the driving circuit may comprise a latching arrangement configured to automatically latch the potential difference applied across the membrane, after insertion of a membrane channel into the membrane, at a value lower than the potential difference applied prior to the insertion of the membrane channel into the membrane.

In such embodiments, the driving circuit and pore insertion circuit 102 are configured to use circuit arrangements whereby upon reduction of the voltage across the membrane (associated with pore insertion causing a drop in the effective resistance across the membrane), the pore insertion circuit 102 is arranged to automatically become disconnected from the membrane. The latching arrangement is configured to be triggered by a fall in the applied potential difference caused by insertion of the membrane channel into the membrane 4.

This embodiment achieves similar advantages to the embodiments of FIGS. 10-12. The risk of the membrane 4 being damaged or popping due to the voltage I_{S}*Rₘ across it is reduced, and the chance of an unwanted second pore inserting into the membrane 4 is reduced. In addition, it is possible that the pore may become fully or partially blocked following insertion. This increases Rₚ and causes the applied potential to further increase. However, because the latching arrangement disconnects the pore insertion circuit 102 after pore insertion, the risk of a high voltage being developed across the membrane 4 in the event of pore blockage is reduced.

An additional advantage is that integrating the means of disconnecting the pore insertion circuit 102 into the pore insertion circuit 102 itself, disconnection may be made instantaneously following pore insertion. This removes the need to use external logic control to periodically disconnect the pore insertion circuit 102 and measure the total membrane resistance RₚRₘ/(Rₘ+Rₚ).

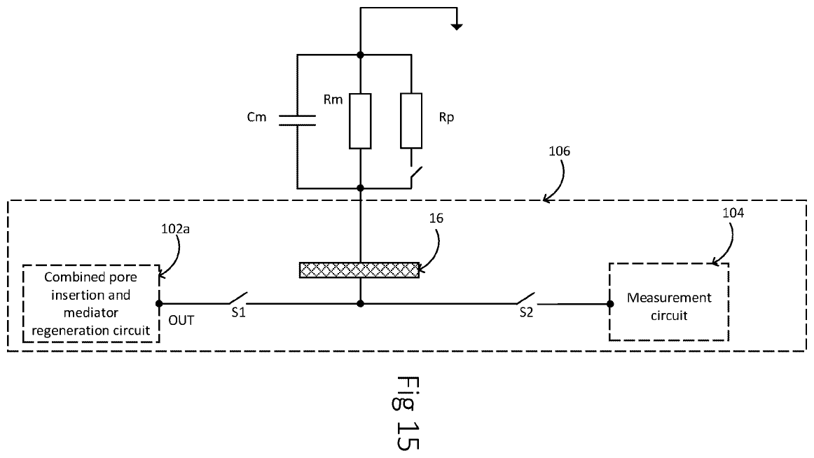

An example pore insertion circuit 102 for achieving disconnection upon pore insertion is shown in FIG. 14. According to operation of this example circuit, upon successful pore insertion, V_{OUT} drops and the output of the comparator U1 transitions from low to high. U2 is also a comparator, whose output will also transition. DFFR is a D-type flip-flop with reset. Upon the output of U2 (connected to input D of DFFR) transitioning high, the output QB of DFFR goes to digital low and latches in this state. Thus, switch S3 (implemented in the example using a transistor) is turned off and the output OUT is isolated from the rest of the circuit. QB will remain low and S3 will remain switched off until DFFR is reset by applying a logic high level to input RESET. Thereby, the pore insertion circuit 102 is automatically disconnected and latches in the disconnected state following membrane channel (pore) insertion into the membrane 4.

Alternatively or additionally to the embodiments above, the driving circuit may be further configured to regenerate a mediator after measurement of an interaction between a molecular entity and a membrane channel inserted into the membrane 4. This is an additional advantage of a nanopore based measurement circuit, i.e. the ability to operate with a reverse bias applied to the membrane 4 in order to regenerate the mediator.

Following pore insertion, the membrane 4 can be used to make measurements of a molecular entity such as a DNA molecule or other biological molecules. The measurements may be, for example, sequencing measurement to sequence a DNA molecule. The measurements are achieved by making measurements of the interaction between the membrane channel and the molecular entity as it passes through the membrane channel. This process can cause depletion of ions in a mediator forming the first and/or second liquids in the first and/or second baths 6, 8. In order to continue measurements of molecular entities, the mediator must be periodically regenerated.

The regeneration of the mediator is performed by applying a potential difference across the membrane 4 that is opposite in polarity to a potential difference applied during the measurement of the interaction between the molecular entity and the membrane channel. This forces a current through the membrane channel opposite to that used during the measurements of the molecular entity.

FIG. 15 shows an embodiment of the apparatus in which the functions of performing pore insertion and mediator regeneration are combined into the same circuitry. The combined pore insertion/regeneration circuit 102a may be provided as part of the integrated electronics of the ASIC 108 described above that is used for membrane channel insertion and measurements of the molecular entity (e.g. molecular sequencing measurements). Any of the circuit arrangements of embodiments described above could be used for the purposes of both pore insertion and mediator regeneration, since they are configured as voltage-limited current sources.

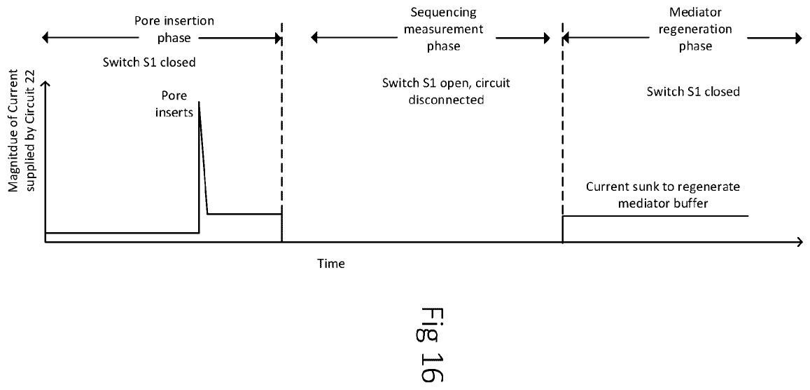

FIG. 16 illustrates a typical operation cycle using the apparatus of FIG. 15. The process operates in three distinct phases.

In the pore insertion phase, occurring before a measurement of the molecular entity (in this example, a sequencing measurement) is performed, switch S1 is closed, S2 is opened and the combined pore insertion/regeneration circuit 102a is configured to supply I_{S} or V_{CLAMP} in accordance with the requirements for pore insertion, as described for previous embodiments.

In the sequencing measurement phase, switch S1 is opened and S2 is closed and the apparatus is configured to measure a sequencing current through the pore, for example using measurement circuit 104. Sequencing for a long time may result in the mediator (the buffer solution forming the first and/or second liquids) becoming depleted, at which point the sequencing measurement may be suspended to regenerate the mediator.

In mediator regeneration mode, switch S2 is opened, S1 is closed and the combined pore insertion/regeneration circuit 102a is configured to source a current I_{S2} (not necessarily equal to I_{S}) to drive the electrochemistry of mediator regeneration. The sign of current I_{S2} should be opposite to the sign of the sequencing current measured in the sequencing measurement. Following regeneration of the mediator buffer a second sequencing measurement phase (not shown) may be employed.

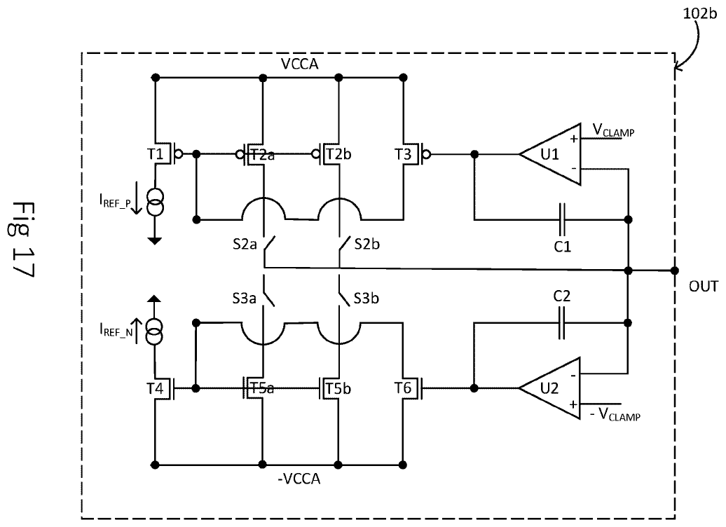

Any of the circuits described above may be modified so that the pore insertion circuit 102 is extended to have the capability to source V_{CLAMP} and I_{S} of either positive or negative sign (102b). For example, FIG. 17 shows such a modification of the circuit of FIG. 7. A symmetrical current mirror comprising T4 and T5 has been added, capable of sinking current I_{SN} when one or more of switches S3a and S3b are closed. A second comparator U2 and negative voltage supply −V_{CLAMP} limit the minimum voltage developed at OUT to −V_{CLAMP}. The example circuit in FIG. 17 then operates essentially as previously described, with the choice of positive or negative polarity of the output voltage and current selected by choice of which of switches S2 or S3 are closed. Accordingly, the circuit may be operated in one polarity for performing pore insertion or another polarity for regeneration of the mediator.

This implementation may be particularly advantageous if the preferred potential for pore insertion is opposite to that for mediator regeneration. The preferred sign of V_{CLAMP} for performing pore insertion may depend on a number of factors including the pH of the first and second liquids (which may be buffer media), and the isoelectric potential of the membrane channel being inserted.

Additionally or alternatively, the driving circuit may be configured to modulate the potential difference applied across the membrane with an AC waveform of smaller amplitude than an average amplitude of the potential difference.

To encourage pore insertion into the membrane 4, it may be favourable under certain conditions to superimpose a small AC potential on top of the DC potential, such that the potential difference applied across the membrane is modulated between V_{CLAMP}+Va and V_{CLAMP}−Va.

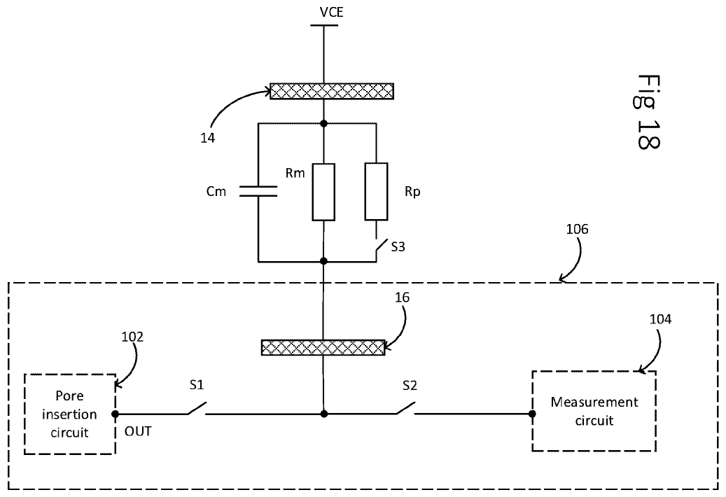

FIG. 18 shows one exemplary arrangement for implementing this. Instead of connecting the first electrode 14 to a fixed DC potential (e.g. 0 Volts) as in previous embodiments, the first electrode is connected to a voltage supply VCE that can provide an AC waveform. Thereby, the modulation of the potential difference is performed by applying the AC waveform to the first electrode 14. This has the advantage of keeping the AC waveform generation separate from the circuitry of the pore insertion circuit 102. However, this is not essential, and in other embodiments, the AC waveform may be applied to the second electrode 16, for example by superimposing the modulation onto V_{CLAMP}.

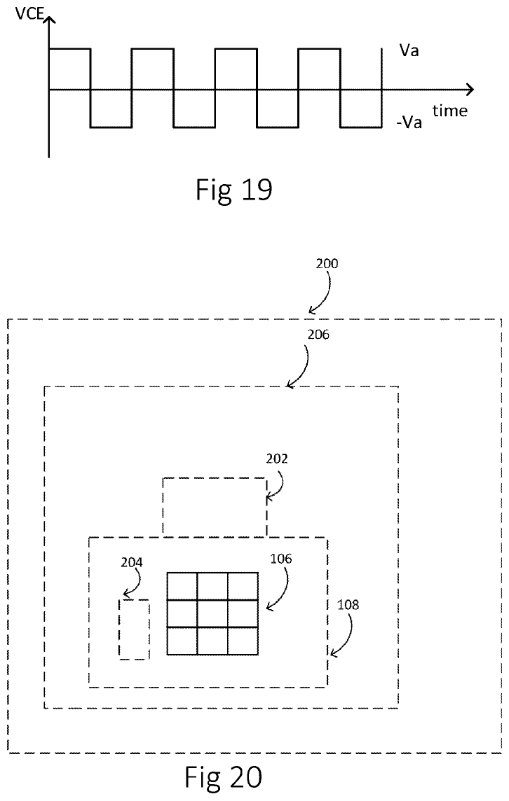

FIG. 19 shows an example of an AC waveform applied to the first electrode 14 in the circuit of FIG. 18. In this example, the waveform is a square wave with amplitude Va. Alternatively, the modulating voltage may be any other AC waveform, for example a sawtooth wave, triangle wave, or sinusoidal waveform. The AC perturbation may have an amplitude in the range 0-100 mV, and/or a frequency in the range 0-10 kHz.

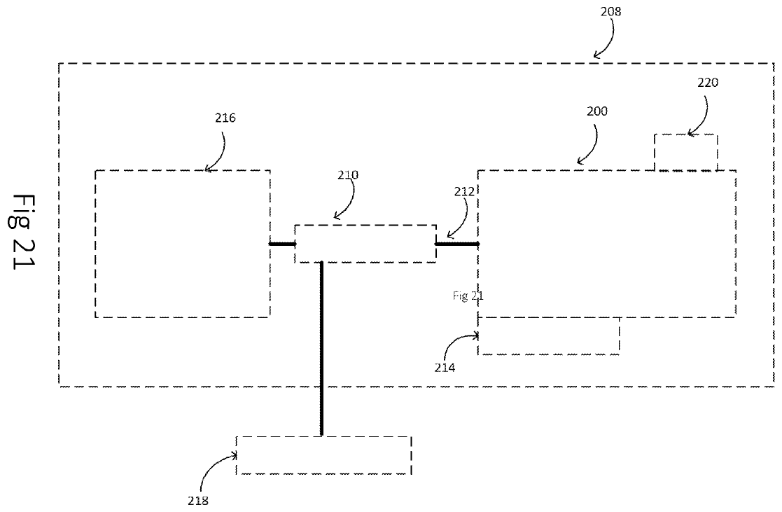

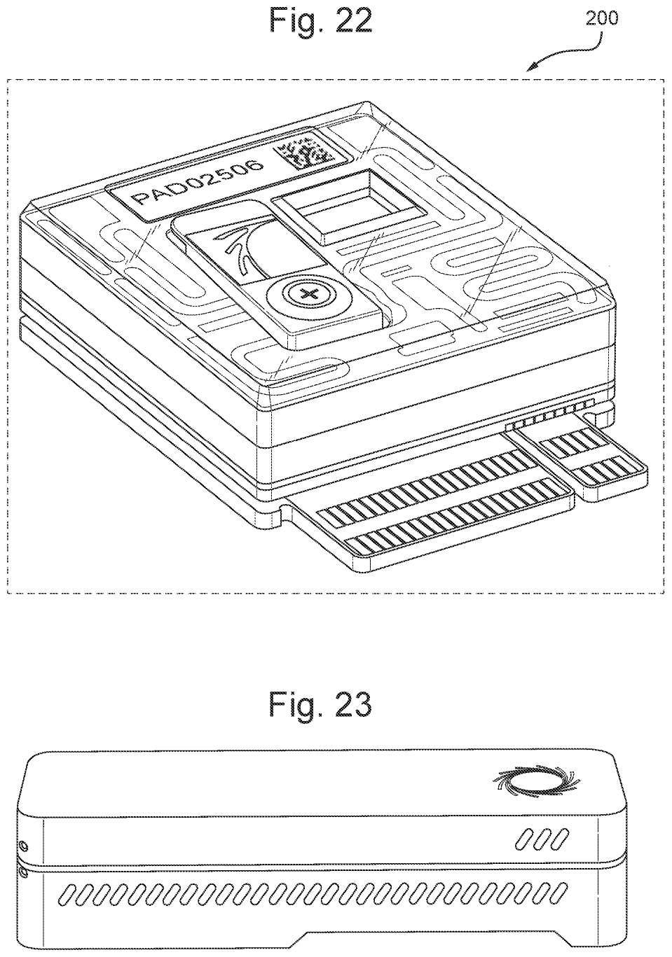

A typical nanopore sequencing system comprises a consumable part, a sequencing flowcell 200, as shown in FIGS. 20 and 22, and a sequencing instrument, shown in FIGS. 21 and 23-25.

The apparatus described above may be provided as part of a sequencing flow cell 200 comprising the application-specific integrated circuit 108 as described above. An example of such a flow cell 200 is shown schematically in FIG. 20.

The flow cell 200 comprises additional electronic components 204, such as the control circuitry 50 configured to control the driving circuit, and other circuitry for driving the ASIC 108 such as voltage references, or non-volatile memory.

The flow cell 200 further comprises a sensor array 202, which may be processed atop or attached to the ASIC 108. The sensor array 202 may be configured to define the first and second baths 6, 8 and support the membrane 4, for example using support structures for defining fluidic wells and supporting the formation of the membrane. The sensor array 202 may further comprise fluidics, and the one or more first electrodes 14, which may be shared between plural pixels 106 in the array of the ASIC 108 as mentioned above.

The flow cell 200 further comprises a housing 206. The housing 206 may be made of any suitable material, for example plastic, and may encase the whole arrangement. The flow cell 200 may further comprise one or more fluid loading ports, and one or more fluid channels fluidly connecting the fluid loading ports to the sensor array 202 and the first and/or second baths 6, 8. The fluid loading ports may be configured to allow loading of reagents, buffers, mediators, and samples (e.g. a molecular entity to be measured) into the flow cell 200, by pipette, “spotting on” or other means. The flow cell 200 may further comprise one or more extraction or waste ports, for removing fluids from the flow cell 200, e.g. used buffer solution.

The flow cell 200 may further comprise an electrical connector for connecting the flow cell 200 to a measurement instrument. In such embodiments, the flow cell 200 may also comprise docking features for securely and/or removably mounting the flow cell 200 into the measurement instrument. Alternatively or additionally, the flow cell 200 may be configured to allow wireless operation not requiring a direct electrical connection to the sequencing instrument. For example, the flow cell 200 may comprise a means for making wireless communication to the measurement instrument, and/or means for obtaining power wirelessly, for example from the measurement instrument.

The flow cell 200 may comprise one or more temperature sensors configured to measure the temperature of all or part of the flow cell 200. For example, a temperature sensor may be provided to measure the temperature of the internal fluidics such as the first and/or second liquids in the first and/or second baths 6, 8. The flow cell 200 may also comprise one or more temperature control elements for heating or cooling all or part of the flow cell 200. For example, a temperature control element may be provided to heat or cool the temperature of the internal fluidics such as the first and/or second liquids in the first and/or second baths 6, 8.

An example of a sequencing instrument 208 in which the sequencing flow cell 200 may be used is shown schematically in FIG. 21. The sequencing instrument 208 may be used with any embodiments of the apparatus described above, not merely those described specifically as sequencing flow cells.

The sequencing instrument 208 may comprise instrumentation electronics 210, configured to interface with and/or control the sequencing flow cell 200. This may be achieved via an electrical connector 212, or via wireless communication means, as mentioned above. The sequencing instrument 208 may further comprise docking features 214 for mechanically docking the sequencing flow cell 200 into the sequencing instrument 208.

The sequencing instrument 208 may comprise integrated computation, such as a processor 216, and/or a means for connecting to external computation 218. The computation (either internal or external) may be configured to control the sequencing instrument 208, and control the sequencing flow cell 200 via the instrumentation electronics 210 to perform functions associated with sequencing measurements. These functions may include analysing measurement data from the flow cell 200 (e.g. calculating a sequence of a nucleotide molecule based on measurements from the apparatuses, for example basecalling of sequencing data from the flow cell 200), controlling the ASIC 108, and specifically the driving circuit of the ASIC, in response to measurement data (e.g. controlling set voltages and currents such as the reference voltage and selected current), controlling the acquisition rate of measurement, setting electronic gain associated with the measurement circuit 1004, performing actions to control ASIC 108 electronics to disconnect, repair, refresh or maintain pixels 106 in an optimum state for sequencing (e.g. disconnecting a pixel 106 if a membrane bursts, modifying applied potentials to compensate for mediator concentration decrease, reversing the applied potential to effect pore unblocking), or switching between different pixels 106 connected to the pixel circuit.

The sequencing instrument 208 may comprise temperature control means 220 for controlling the temperature of the sequencing flow cell 200, and specifically the temperature of the first and second liquids. The temperature control means 220 may comprise one or more of heater elements, cooling elements, and control electronics. The control electronics may be based on PID control, and may interface with temperature control elements in the flow cell 200 itself, if they are present.

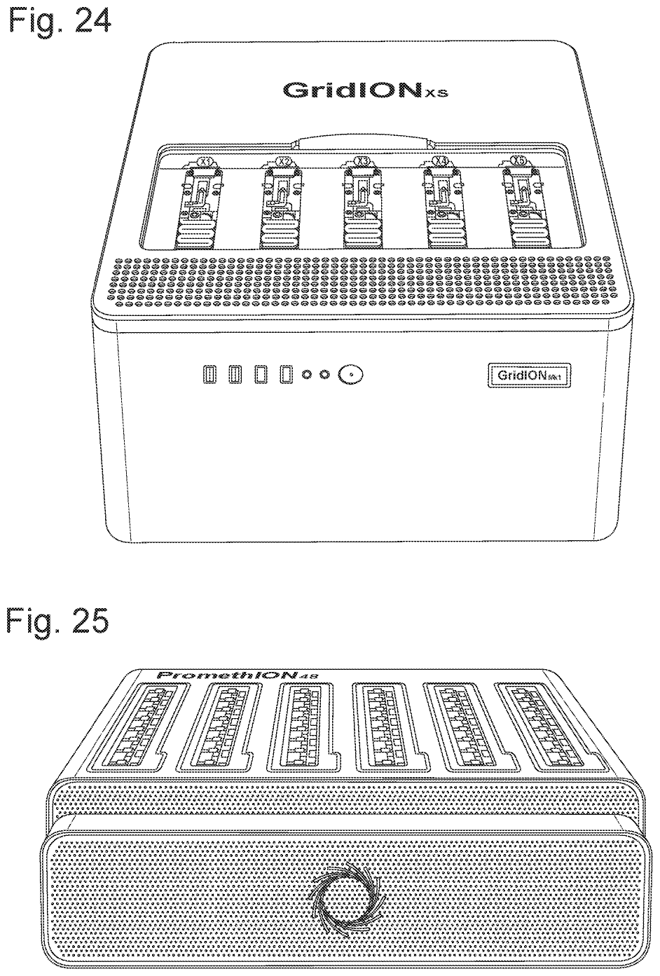

Depending on the application, the sequencing instrument 208 may be configured to connect to a single flow cell 200, or to multiple flow cells 200 simultaneously. For example Oxford Nanopore products comprise the MinION (1 flow cell per sequencing instrument, FIG. 23), GridION (5 flow cells per Sequencing Instrument, FIG. 24) and PromethION (24 or 48 flow cells per sequencing instrument, FIG. 25).

Embodiments of the invention have been described with reference to the apparatus above, but the invention may also be embodied in a method for controlling insertion of a membrane channel into a membrane.

There is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises controlling a driving circuit to apply a potential difference across the membrane via the first and second electrodes to promote insertion of a membrane channel into the membrane from the first liquid or the second liquid, wherein: the applied potential difference prior to insertion of a membrane is defined by a reference voltage; and the applied potential difference after insertion of a membrane is defined by a selected current provided by a current source and a resistance between the first and second electrodes, a voltage limit of the current source being independent of the reference voltage, wherein the applied potential difference after insertion of the membrane channel is lower than the applied potential difference prior to insertion of the membrane channel.

The method may further comprise controlling the driving circuit to apply progressively increasing potential differences across the membrane. Alternatively or additionally, the method may further comprise periodically measuring one or more electrical properties of the membrane; and blocking subsequent applying of the potential difference across the membrane in response to the one or more electrical properties.

There is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises: controlling a driving circuit to apply a potential difference across the membrane via the first and second electrodes to promote insertion of a membrane channel into the membrane from the first liquid or the second liquid, wherein: the driving circuit is configured to automatically lower the potential difference in response to the insertion of the membrane channel into the membrane; and the driving circuit comprises a latching arrangement configured to automatically latch the potential difference applied across the membrane, after insertion of a membrane channel into the membrane, at a value lower than the potential difference applied prior to the insertion of the membrane channel into the membrane.

There is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises controlling a driving circuit to: apply a potential difference across the membrane via the first and second electrodes to promote insertion of a membrane channel into the membrane from the first liquid or the second liquid; and regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

There is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises controlling a driving circuit to apply a potential difference across the membrane via the first and second electrodes to promote insertion of a membrane channel into the membrane from the first liquid or the second liquid; and modulating the potential difference applied across the membrane with an AC waveform of smaller amplitude than an average amplitude of the potential difference.

There is provided a method for controlling insertion of a membrane channel into a membrane using an apparatus comprising: a first bath for holding a first liquid in contact with a first surface of the membrane; a second bath for holding a second liquid in contact with a second surface of the membrane, wherein the membrane separates the first and second liquids; a first electrode configured to contact the first liquid; a second electrode configured to contact the second liquid, wherein the method comprises: controlling a driving circuit to apply a potential difference across the membrane via the first and second electrodes to promote insertion of a membrane channel into the membrane from the first liquid or the second liquid measuring one or more electrical properties of the membrane; and setting or adjusting the potential difference applied across the membrane, before insertion of a membrane channel into the membrane, based on the measured one or more electrical properties.

The methods may be modified with any of the features as described above for the apparatus as appropriate, and as would be apparent to the skilled person.

1. An apparatus for controlling insertion of a membrane channel into a membrane, comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid; and
a driving circuit configured to apply a potential difference across the membrane via the first and second electrodes to promote insertion of a membrane channel into the membrane from the first liquid or the second liquid, wherein:
the driving circuit is configured such that:
the applied potential difference prior to insertion of a membrane channel is defined by a reference voltage; and
the applied potential difference after insertion of a membrane channel is defined by a selected current provided by a current source and a resistance between the first and second electrodes, a voltage limit of the current source being independent of the reference voltage, wherein the applied potential difference after insertion of the membrane channel is lower than the applied potential difference prior to insertion of the membrane channel.

2. The apparatus of claim 1, wherein the driving circuit comprises at least one power rail defining the reference voltage and at least one diode in series between the at least one power rail and the first or second electrode, the at least one power rail and at least one diode being configured to limit the applied potential difference to be no greater than the reference voltage.

3. The apparatus of claim 2, wherein the at least one power rail and at least one diode are configured to limit a magnitude of a voltage applied at the first electrode or the second electrode to be no greater than the reference voltage.

4. The apparatus of claim 1, wherein the driving circuit comprises a comparator configured to compare the reference voltage with a voltage at the first or second electrode, an output from the comparator determining whether the applied potential difference is defined by the reference voltage or by the selected current.

5. The apparatus of any preceding claim, wherein a voltage at the first electrode is held constant, and the applied potential difference is controlled by controlling a voltage at the second electrode.

6. The apparatus of any preceding claim, wherein the selected current is in the range of 50-300 pA and, optionally, a resistance of an inserted membrane channel is in the range of 0.1-10 GOhms.

7. The apparatus of any preceding claim, wherein the driving circuit is configured to allow the selected current to be selected from a plurality of available current values, preferably by logic control.

8. The apparatus of any preceding claim, wherein the driving circuit is configured to apply progressively increasing potential differences across the membrane.

9. The apparatus of any preceding claim, wherein the apparatus is configured to periodically measure one or more electrical properties of the membrane and to block subsequent applying of the potential difference across the membrane by the driving circuit in response to the one or more electrical properties being indicative of a membrane channel having been inserted into the membrane.

10. The apparatus of claim 9, wherein:
the apparatus comprises a measurement circuit configured to perform the periodic measuring of the one or more electrical properties of the membrane; and
the apparatus is configured to switch from a driving state to a measuring state for each measurement of the one or more electrical properties of the membrane, the driving state being such that the driving circuit is electrically connected to the first electrode or second electrode in such a way as to be able to apply the potential difference across the membrane, and the measuring state being such that the driving circuit is not electrically connected to the first electrode or second electrode in such a way as to be able to apply the potential difference across the membrane.

11. The apparatus of claim 10, wherein the apparatus is configured to block the subsequent application of the potential difference by not switching from the measuring state back to the driving state.

12. The apparatus of any preceding claim, wherein the driving circuit comprises a latching arrangement configured to automatically latch the potential difference applied across the membrane, after insertion of a membrane channel into the membrane, at a value lower than the potential difference applied prior to the insertion of the membrane channel into the membrane.

13. An apparatus for controlling insertion of a membrane channel into a membrane, comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid; and
a driving circuit configured to apply a potential difference across the membrane via the first and second electrodes to promote insertion of a membrane channel into the membrane from the first liquid or the second liquid, wherein:
the driving circuit is configured to automatically lower the potential difference in response to the insertion of the membrane channel into the membrane; and
the driving circuit comprises a latching arrangement configured to automatically latch the potential difference applied across the membrane, after insertion of a membrane channel into the membrane, at a value lower than the potential difference applied prior to the insertion of the membrane channel into the membrane.

14. The apparatus of claim 12 or 13, wherein the latching arrangement is configured to be triggered by a fall in the applied potential difference caused by insertion of the membrane channel into the membrane.

15. The apparatus of any preceding claim, wherein the driving circuit is further configured to regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

16. An apparatus for controlling insertion of a membrane channel into a membrane, comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid; and
a driving circuit configured to apply a potential difference across the membrane via the first and second electrodes to promote insertion of a membrane channel into the membrane from the first liquid or the second liquid, wherein:
the driving circuit is further configured to regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

17. The apparatus of any preceding claim, wherein the driving circuit is configured to modulate the potential difference applied across the membrane with an AC waveform of smaller amplitude than an average amplitude of the potential difference.

18. An apparatus for controlling insertion of a membrane channel into a membrane, comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid; and
a driving circuit configured to apply a potential difference across the membrane via the first and second electrodes to promote insertion of a membrane channel into the membrane from the first liquid or the second liquid, wherein:
the driving circuit is configured to modulate the potential difference applied across the membrane with an AC waveform of smaller amplitude than an average amplitude of the potential difference.

19. The apparatus of claim 17 or 18, wherein the AC waveform has an amplitude in the range of 0-100 mV and/or a frequency in the range of 0-10 kHz.

20. The apparatus of any of claims 17 to 19, wherein the modulation of the potential difference is performed by applying the AC waveform to the first electrode.

21. The apparatus of any preceding claim, configured to:
measure one or more electrical properties of the membrane; and
set or adjust the potential difference applied across the membrane, before insertion of a membrane channel into the membrane, based on the measured one or more electrical properties.

22. An apparatus for controlling insertion of a membrane channel into a membrane, comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid; and
a driving circuit configured to apply a potential difference across the membrane via the first and second electrodes to promote insertion of a membrane channel into the membrane from the first liquid or the second liquid, wherein:
the apparatus is configured to:
measure one or more electrical properties of the membrane; and
set or adjust the potential difference applied across the membrane, before insertion of a membrane channel into the membrane, based on the measured one or more electrical properties.

23. The apparatus of any preceding claim, wherein the driving circuit is configured to promote insertion of a membrane channel into a membrane simultaneously at a plurality of different pixels, each pixel being associated with a first electrode and a second electrode driven by the driving circuit.

24. The apparatus of claim 23, wherein the driving circuit is configured to apply a common potential difference to pixels in which a membrane channel has not yet inserted into the membrane.

25. The apparatus of claim 24, wherein the driving circuit is configured to progressively increase the common potential difference and to control the progressive increase based on detection of membrane channel insertions in other pixels.

26. The apparatus of claim 25, wherein the driving circuit is configured to control the progressive increase based on a detected rate of membrane channel insertion.

27. The apparatus of claim 26, wherein the progressive increase comprises a series of steps and the driving circuit is configured to initiate a step to a higher potential difference when the detected rate of membrane channel insertion falls below a predetermined threshold.

28. The apparatus of any of claims 23-27, wherein the driving circuit is configured to:
measure one or more electrical properties of the membrane in each of the pixels; and
set or adjust the potential difference applied across the membrane in each pixel, before insertion of a membrane channel into the membrane of the pixel, based on the measured one or more electrical properties.

29. The apparatus of any of claims 23 to 28, wherein each pixel is associated with a respective pair of first and second electrodes driven by the driving circuit.

30. The apparatus of any of claims 23 to 28, wherein two or more of the plurality of pixels are associated with respective second electrodes and share a common first electrode.

31. The apparatus of claim 30, wherein the two or more of the plurality of pixels are associated with the first bath and a plurality of respective second baths, the common first electrode being configured to contact the first liquid in the first bath, and the respective second electrodes being configured to contact the second liquid in the respective second baths.

32. The apparatus of any of claims 23 to 31, wherein the second electrodes are individually addressable by the driving circuit.

33. The apparatus of claim 32, wherein the pixels are arranged in a two-dimensional grid, and the second electrode associated with each pixel is connected to the second electrodes associated with other pixels on the same row or column of the grid, such that each second electrode is individually addressable using a combination of a row and column.

34. The apparatus of any of claims 23 to 33, wherein the second electrodes are each provided at the base of a respective second bath, the driving circuit is provided in an integrated circuit integral with the second bath, and the second electrodes are electrically connected to the driving circuit.

35. The apparatus of any of claims 23 to 33, wherein the second electrodes are detachable from the driving circuit.

36. The apparatus of any preceding claim, wherein the driving circuit is provided by an application-specific integrated circuit.

37. A sequencing flow cell comprising the apparatus of any preceding claim, and further comprising:
a control circuit configured to control the driving circuit;
a sensor array configured to define the first and second baths and support the membrane;
a housing comprising one or more fluid loading ports;
one or more fluid channels fluidly connecting the fluid loading ports to the first and/or second baths; and
an electrical connector for connecting the flow cell to a measurement instrument.

38. A sequencing instrument comprising one or more of the apparatus of any of claims 1-36, and further comprising:
instrumentation electronics configured to interface with the one or more apparatuses;
a processor configured to control the driving circuits of the one or more apparatuses via the instrumentation electronics, and to calculate a sequence of a nucleotide molecule based on measurements from the apparatuses; and
optionally further comprising temperature control means configured to control the temperature of the first and second liquids in the one or more apparatuses.

39. The sequencing instrument of claim 38, wherein:
the one or more apparatuses are provided by sequencing flow cells according to claim 37;
the electrical connector of each of the one or more sequencing flow cells is configured to connect to the instrumentation electronics; and
the processor is configured to control the driving circuits of the one or more flow cells via the control circuits of the respective flow cells.

40. A method for controlling insertion of a membrane channel into a membrane using an apparatus comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid,
wherein the method comprises
controlling a driving circuit to apply a potential difference across the membrane via the first and second electrodes to promote insertion of a membrane channel into the membrane from the first liquid or the second liquid, wherein:
the applied potential difference prior to insertion of a membrane channel is defined by a reference voltage; and
the applied potential difference after insertion of a membrane channel is defined by a selected current provided by a current source and a resistance between the first and second electrodes, a voltage limit of the current source being independent of the reference voltage, wherein the applied potential difference after insertion of the membrane channel is lower than the applied potential difference prior to insertion of the membrane channel.

41. The method of claim 40, further comprising controlling the driving circuit to apply progressively increasing potential differences across the membrane.

42. The method of claim 40 or 41, further comprising:
periodically measuring one or more electrical properties of the membrane; and
blocking subsequent applying of the potential difference across the membrane in response to the one or more electrical properties being indicative of a membrane channel having been inserted into the membrane.

43. A method for controlling insertion of a membrane channel into a membrane using an apparatus comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid,
wherein the method comprises:
controlling a driving circuit to apply a potential difference across the membrane via the first and second electrodes to promote insertion of a membrane channel into the membrane from the first liquid or the second liquid, wherein:
the driving circuit is configured to automatically lower the potential difference in response to the insertion of the membrane channel into the membrane; and
the driving circuit comprises a latching arrangement configured to automatically latch the potential difference applied across the membrane, after insertion of a membrane channel into the membrane, at a value lower than the potential difference applied prior to the insertion of the membrane channel into the membrane.

44. A method for controlling insertion of a membrane channel into a membrane using an apparatus comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid,
wherein the method comprises controlling a driving circuit to:
apply a potential difference across the membrane via the first and second electrodes to promote insertion of a membrane channel into the membrane from the first liquid or the second liquid; and
regenerate a mediator after measurement of an interaction between a molecular entity and a membrane channel inserted into the membrane, the regeneration of the mediator being performed by applying a potential difference across the membrane that is opposite in polarity to a potential difference applied during the measurement of the interaction between the molecular entity and the membrane channel.

45. A method for controlling insertion of a membrane channel into a membrane using an apparatus comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid,
wherein the method comprises
controlling a driving circuit to apply a potential difference across the membrane via the first and second electrodes to promote insertion of a membrane channel into the membrane from the first liquid or the second liquid; and
modulating the potential difference applied across the membrane with an AC waveform of smaller amplitude than an average amplitude of the potential difference.

46. A method for controlling insertion of a membrane channel into a membrane using an apparatus comprising:
a first bath for holding a first liquid in contact with a first surface of the membrane;
a second bath for holding a second liquid in contact with a second surface of the membrane, wherein the membrane separates the first and second liquids;
a first electrode configured to contact the first liquid;
a second electrode configured to contact the second liquid,
wherein the method comprises:
controlling a driving circuit to apply a potential difference across the membrane via the first and second electrodes to promote insertion of a membrane channel into the membrane from the first liquid or the second liquid
measuring one or more electrical properties of the membrane; and
setting or adjusting the potential difference applied across the membrane, before insertion of a membrane channel into the membrane, based on the measured one or more electrical properties.
